(19) United States
(12) Patent Application Publication
(10) Pub. No.: US 2021/0317157 A1
(43) Pub. Date: Oct. 14, 2021

(54) Substituted Alloxazine Nucleosides and Nucleotides, and Methods of Making Same

(71) Applicant: THE REGENTS OF THE UNIVERSITY OF COLORADO, A BODY CORPORATE, DENVER, CO (US)

(72) Inventors: Robert D. Kuchta, Boulder, CO (US); Ayman Alawneh, Boulder, CO (US)

(21) Appl. No.: 17/224,728

(22) Filed: Apr. 7, 2021

Related U.S. Application Data
(60) Provisional application No. 63/007,001, filed on Apr. 8, 2020.

(51) Int. Cl. C07H 19/23; C07D 471/04; C07D 487/04; C07H 21/02; C07H 21/04

(52) U.S. Cl. CPC C07H 19/23; C07D 471/04; C07H 21/04; C07H 21/02; C07D 487/04

(57) ABSTRACT

The present disclosure includes nucleosides and/or nucleotides comprising an optionally substituted alloxazine (also known as benzo[g]pteridine-2,4(1H,3H)-dione, or isoalloxazine) at the anomeric position.

### CROSS-REFERENCE TO RELATED APPLICATIONS

This application claims priority under 35 U.S.C. § 119(e) to U.S. Provisional Patent Application No. 63/007,001, filed Apr. 8, 2020, the entire contents of which are incorporated herein by reference.

### STATEMENT REGARDING FEDERALLY SPONSORED RESEARCH

This invention was made with government support under grant number W911NF-16-1-1234 awarded by the U.S. Army Research Office. The government has certain rights in the invention.

### BACKGROUND

Nucleic acids are biopolymers essential to all known forms of life. The term nucleic acid is the overall name for DNA (deoxyribonucleic acid) and RNA (ribonucleic acid). Nucleic acids are composed of nucleotides, which generally comprise a 5-carbon sugar, a phosphate group, and a nitrogenous base. If the sugar is the compound ribose, the polymer is RNA. If the sugar is derived from ribose as 2′-deoxyribose, the polymer is DNA.

Nucleic acids function to create, encode, and store genetic information of every living cell of every organism. Further, nucleic acids transmit and express that genetic information inside and outside the cell nucleus—to the interior operations of the cell and ultimately to the next generation of each living organism. The encoded genetic information is contained in the nucleic acid sequence, comprising the “ladder-step” ordering of nucleotides within the molecules of RNA and DNA. The nucleotides are assembled into chains of base pairs selected from the five canonical nucleobases: adenine, cytosine, guanine, thymine, and uracil. Thymine occurs only in DNA and uracil only in RNA.

As nucleic acids are the basis of life, there is a need in the art to identify novel nucleic acid building blocks that allow for shedding light and/or altering the biological role of nucleic acids in a rational manner. The present disclosure addresses this need.

### SUMMARY OF THE DISCLOSURE

In one aspect, the present disclosure relates to a compound of formula (III):

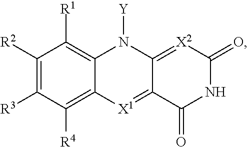

or a salt, solvate, tautomer, N-oxide, enantiomer, or diastereoisomer thereof, wherein Y, X¹, X², R¹, R², R³, and R⁴ are described elsewhere herein. In certain embodiments, each of R¹, R², R³, and R⁴ are H. In certain embodiments, Y is

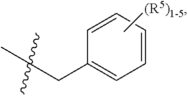

wherein at least one R⁵ is methoxy. In certain embodiments, the compound is selected from the group consisting of:

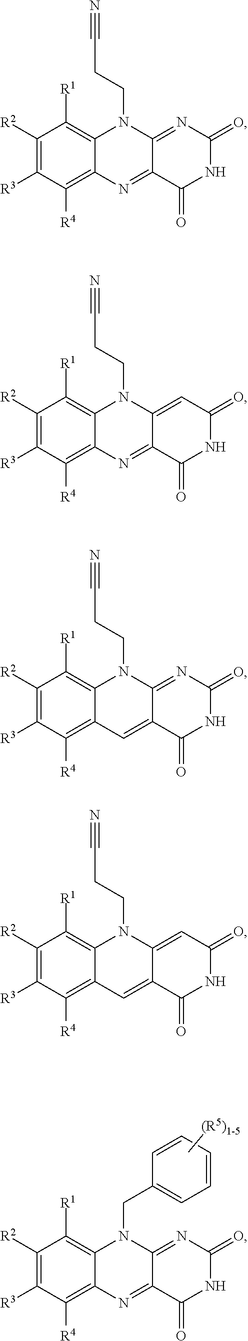

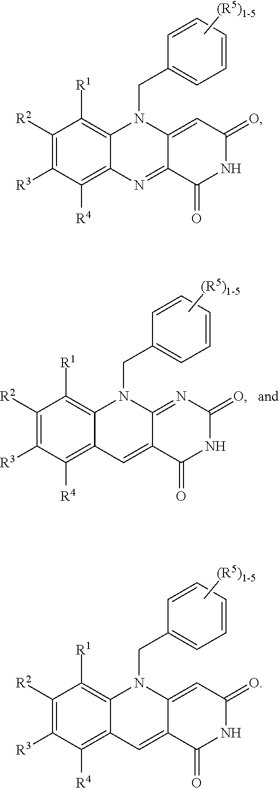

In another aspect, the present disclosure relates to a method of preparing a derivatized nucleoside, the method comprising reacting the compound of (III), or a salt, solvate, tautomer, N-oxide, enantiomer, or diastereoisomer thereof, with a sugar donor comprising an anomeric leaving group. In certain embodiments, the anomeric leaving group is a halogen, ester, amide, mesylate, triflate, sulfide, sulfoxide, or sulfone. In certain embodiments, the anomeric leaving group is an ester and the reaction is performed in the presence of a silylating agent and a Lewis acid. In certain embodiments, the sugar is a ribose or ribose derivative. In certain embodiments, the sugar is a deoxyribose or deoxyribose derivative. In certain embodiments, the sugar donor is one of the following:

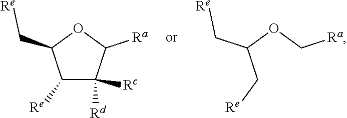

wherein R^{a}, R^{c}, R^{d} and R^{e} are described elsewhere herein. In certain embodiments, the resulting derivatized nucleoside is subjected to deprotecting conditions that allow for replacement of Y with a hydrogen. In certain embodiments, Y is —CH₂CH₂CN and the deprotecting conditions comprise treatment with a base. In certain embodiments, Y is

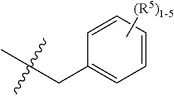

and the deprotecting conditions comprise treatment with an oxidizing agent. In certain embodiments, the derivatized nucleoside is further converted to a phosphoramidite derivative.

In yet another aspect, the present disclosure relates to a nucleic acid wherein at least one base is:

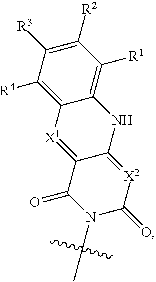

wherein X¹, X², R¹, R², R³, and R⁴ are described elsewhere herein. In certain embodiments, the nucleic acid is a ribonucleic acid. In other embodiments, the nucleic acid is a deoxyribonucleic acid.

### DETAILED DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

In one aspect, the present disclosure relates to nucleosides and/or nucleotides comprising an optionally substituted alloxazine (also known as benzo[g]pteridine-2,4(1H,3H)-dione, or isoalloxazine) at the anomeric position, such as but not limited to the non-limiting example shown herein:

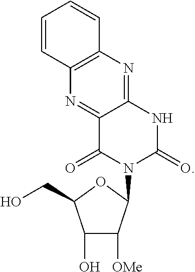 [SMILES: COC1C(O)[C@@H](CO)O[C@H]1n1c(=O)[nH]c2nc3ccccc3nc2c1=O]

In certain embodiments, the optionally substituted alloxazine group is attached by N-3 position to the anomeric carbon of the nucleoside sugar. The disclosure contemplates any nucleoside sugar known in the art, such as but limited to ribose, deoxyribose, or derivatives or analogues thereof. In certain embodiments, the alloxazine is attached by the N-3 position to the anomeric carbon of the nucleoside sugar, and the resulting nucleoside/nucleotide is a pyrimidine analogue. In other embodiments, the resulting nucleoside/nucleotide can base pair with a purine nucleoside/nucleotide. In yet other embodiments, the resulting nucleoside/nucleotide is a thymidine nucleoside/nucleotide analogue. In yet other embodiments, the resulting nucleoside/nucleotide is incorporated in a nucleic acid and used to study the base pairing properties and/or stability and/or biological properties of the resulting nucleic acid.

A non-limiting example of an illustrative synthesis of a nucleoside contemplated in the present disclosure follows. The Bz protective groups in the sugar reagent can be replaced with other protective groups known in the art.

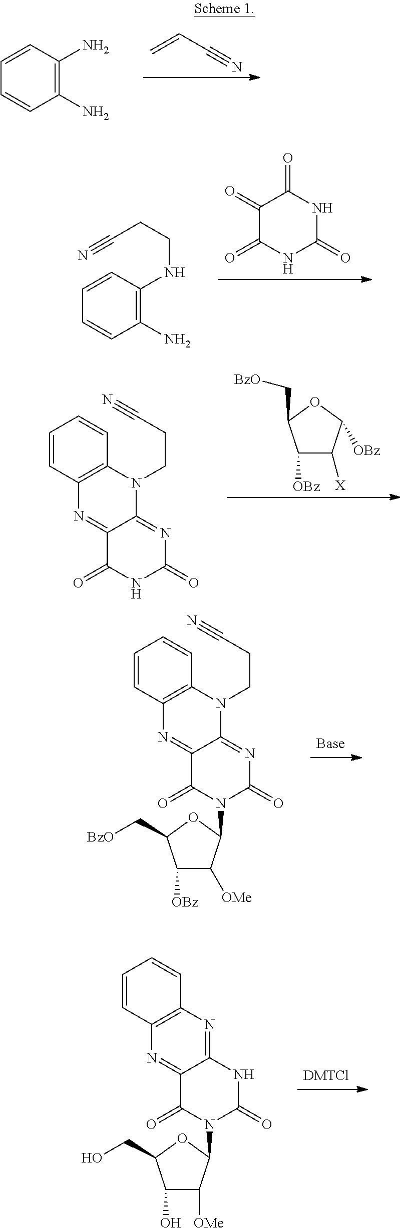

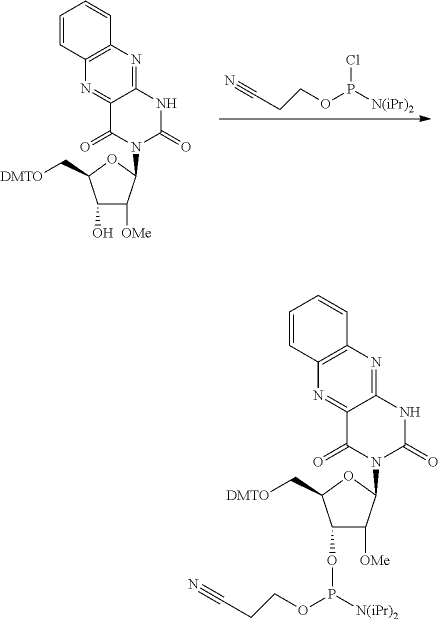

Scheme 1 comprises the synthesis scheme for synthesizing 3-(alloxazine)-5′-dimethoxytrityl-2′-methoxy-2′-deoxyribose phosphoramidite, which can then be incorporated into DNA using standard phosphoramidite synthesis. A similar approach can be used for other nucleosides or nucleotides, by using varying alloxazine and sugar blocking blocks.

The nucleosides/nucleotides of the present disclosure can be incorporated into nucleic acids under mild conditions, and the resulting nucleic acid can be purified under standard conditions. The disclosure contemplates use of (de)protection steps and purification procedures that are compatible with the nucleoside/nucleotide molecule, including the alloxazine group.

Alloxazine is redox active, and can accept and donate electrons reversibly. This is in contrast with the natural nucleosides found in nucleic acids. The natural nucleosides are nominally redox reactive, but their redox reactions are not reversible and may lead to permanent nucleic acid damage.

The nucleic acid incorporating the alloxazine group can reversibly accept electrons from exogenously added reducing agents (such as, but not limited to, dithionite), and donate electrons to exogenously added oxidizing agents (such as, but not limited to, oxygen). In certain embodiments, the nucleic acids of the present disclosure, incorporating at least one alloxazine nucleoside/nucleotide, can bind to a complementary nucleic acid. In other embodiments, the redox-activated alloxazine can promote irreversible redox modification of the complementary nucleic acid strand.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type might be abnormal for a different cell or tissue type.

A disease or disorder is “alleviated” if the severity of a symptom of the disease or disorder, the frequency with which such a symptom is experienced by a patient, or both, is reduced.

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Eₘₐₓ) achieved within an assay.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate, sulfone or amide linkages, and combinations of such linkages. The term nucleic acid also specifically includes nucleic acids composed of bases other than the five biologically occurring bases (adenine, guanine, thymine, cytosine and uracil), as well as peptide nucleic acid (PNA). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “oligonucleotide” typically refers to short polynucleotides, generally no greater than about 200 nucleotides. It will be understood that when a nucleotide sequence is represented by a DNA sequence (i.e., A, T, G, C), this also includes an RNA sequence (i.e., A, U, G, C) in which “U” replaces “T.”

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the disclosure include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

A “polynucleotide” means a single strand or parallel and anti-parallel strands of a nucleic acid. Thus, a polynucleotide may be either a single-stranded or a double-stranded nucleic acid. In the context of the present disclosure, the following abbreviations for the commonly occurring nucleic acid bases are used. “A” refers to adenosine, “C” refers to cytidine, “G” refers to guanosine, “T” refers to thymidine, and “U” refers to uridine.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the disclosure (alone or in combination with another pharmaceutical agent), to a patient, or application or administration of a therapeutic agent to an isolated tissue or cell line from a patient (e.g., for diagnosis or ex vivo applications), who has a condition contemplated herein and/or a symptom of a condition contemplated herein, with the purpose to cure, heal, alleviate, relieve, alter, remedy, ameliorate, improve or affect a condition contemplated herein and/or the symptoms of a condition contemplated herein. Such treatments may be specifically tailored or modified, based on knowledge obtained from the field of pharmacogenomics.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(═O)N(R)₂, CN, CF₃, OCF₃, R, C(═O), methylenedioxy, ethylenedioxy, N(R)₂, SR, S(═O)R, S(═O)₂R, S(═O)₂N(R)₂, SO₃R, C(═O)R, C(═O)C(═O)R, C(═O)CH₂C(═O)R, C(═S)R, C(═O)OR, OC(═O)R, C(═O)N(R)₂, OC(═O)N(R)₂, C(═S)N(R)₂, (CH₂)₀₋₂N(R)C(═O)R, (CH₂)₀₋₂N(R)N(R)₂, N(R)N(R)C(═O)R, N(R)N(R)C(═O)OR, N(R)N(R)C(═O)N(R)₂, N(R)S(═O)₂R, N(R)S(═O)₂N(R)₂, N(R)C(═O)OR, N(R)C(═O)R, N(R)C(═S)R, N(R)C(═O)N(R)₂, N(R)C(═S)N(R)₂, N(C(═O)R)—C(═O)R, N(OR)R, C(═NH)N(R)₂, C(═O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(═O)N(R)₂, CN, NO, NO₂, ONO₂, azido, CF₃, OCF₃, R, O (oxo), S (thiono), C(═O), S(═O), methylenedioxy, ethylenedioxy, N(R)₂, SR, S(═O)R, S(═O)₂R, S(═O)₂N(R)₂, SO₃R, C(═O)R, C(═O)C(═O)R, C(═O)CH₂C(═O)R, C(═S)R, C(═O)OR, OC(═O)R, C(═O)N(R)₂, OC(═O)N(R)₂, C(═S)N(R)₂, (CH₂)₀₋₂N(R)C(═O)R, (CH₂)₀₋₂N(R)N(R)₂, N(R)N(R)C(═O)R, N(R)N(R)C(═O)OR, N(R)N(R)C(═O)N(R)₂, N(R)S(═O)₂R, N(R)S(═O)₂N(R)₂, N(R)C(═O)OR, N(R)C(═O)R, N(R)C(═S)R, N(R)C(═O)N(R)₂, N(R)C(═S)N(R)₂, N(C(═O)R)—C(═O)R, N(OR)R, C(═NH)N(R)₂, C(═O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group. Illustrative examples of cycloalkyl groups include, but are not limited to, the following moieties.

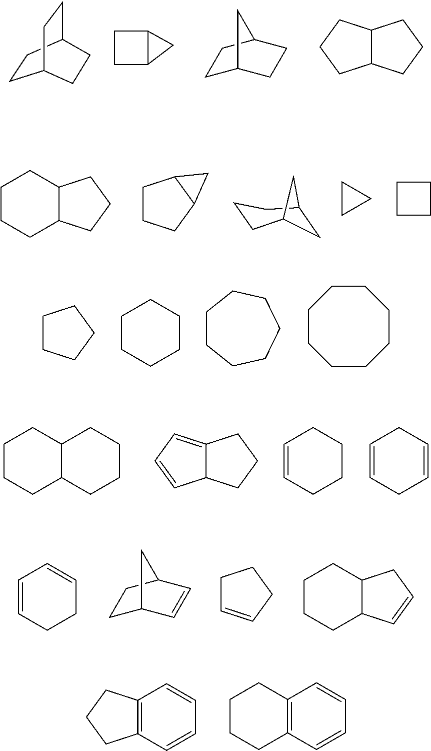

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group.

The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein. Non-limiting examples of heterocycloalkyl groups include:

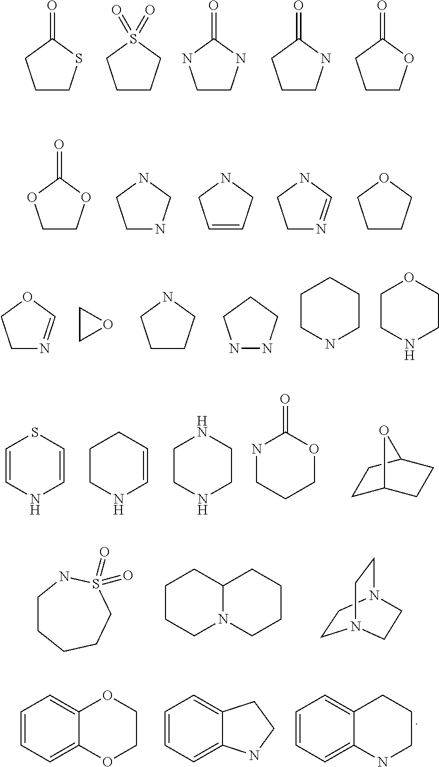

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein. Non-limiting examples of heteroaryl groups include the following moieties:

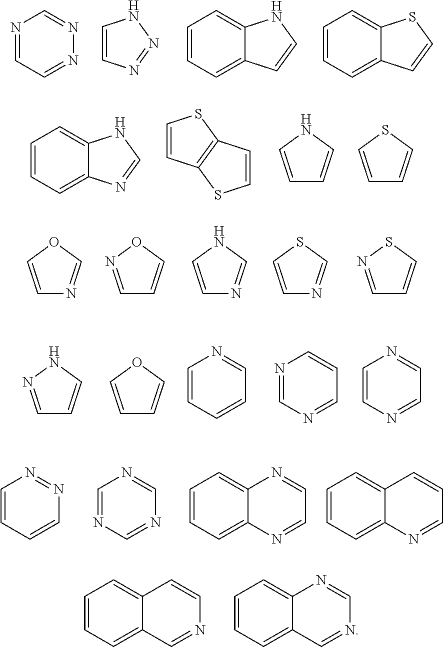

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxycyclohexyl)ethyl, 2-(2,3-epoxycylopentyl)ethyl, 2-(4-methyl-3,4-epoxycyclohexyl)propyl, 2-(3,4-epoxy-3-methylcylohexyl)-2-methylethyl, and 5,6-epoxyhexyl.

The term “monovalent” as used herein refers to a substituent connecting via a single bond to a substituted molecule. When a substituent is monovalent, such as, for example, F or Cl, it is bonded to the atom it is substituting by a single bond.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

### Compounds

The compounds described herein can be synthesized using techniques well-known in the art of organic synthesis. The starting materials and intermediates required for the synthesis can be obtained from commercial sources or synthesized according to methods known to those skilled in the art.

In one aspect, the present disclosure contemplates a compound of Formula (I), or a salt, solvate, tautomer, N-oxide, enantiomer, or diastereoisomer thereof:

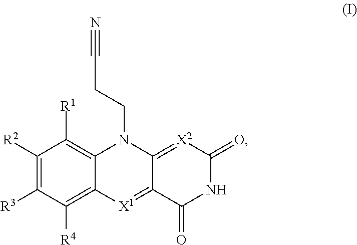

wherein:

X¹ is CH or N;

X² is CH or N;

R¹, R², R³, and R⁴ are independently selected from the group consisting of R, CF₃, OR, OCF₃, C(═O)R, C(═S)R, CN, NO, NO₂, azido, F, Cl, Br, I, N(R)₂, SR, S(═O)R, S(═O)₂R, SO₃R, S(═O)₂N(R)₂, N(R)S(═O)₂R, N(R)S(═O)₂N(R)₂, C(═O)C(═O)R, C(═O)CH₂C(═O)R, C(═O)OR, OC(═O)R, C(═O)N(R)₂, OC(═O)N(R)₂, C(═S)N(R)₂, (CH₂)₀₋₂N(R)C(═O)R, (CH₂)₀₋₂N(R)N(R)₂, N(R)N(R)C(═O)R, N(R)N(R)C(═O)OR, N(R)N(R)C(═O)N(R)₂, N(R)C(═O)R, N(R)C(═O)OR, N(R)C(═S)R, N(R)C(═O)N(R)₂, N(R)C(═S)N(R)₂, N(C(═O)R)—C(═O)R, N(OR)R, C(═NH)N(R)₂, C(═O)N(OR)R, and C(═NOR)R, or two adjacent groups of R¹-R⁴ combine to form methylenedioxy or ethylenedioxy,
or R¹ and R² combine to form an optionally substituted arylene or heteroarylene group (which is fused to the phenyl group to which R¹ and R² are bound) and/or R² and R³ combine to form an optionally substituted arylene or heteroarylene group (which is fused to the phenyl group to which R² and R³ are bound) and/or R³ and R⁴ combine to form an optionally substituted arylene or heteroarylene group (which is fused to the phenyl group to which R³ and R⁴ are bound);

and

each occurrence of R is independently H or an optionally substituted carbon-based moiety, such as but not limited to optionally substituted alkyl or optionally substituted cycloalkyl.

In certain embodiments, the compound of formula (I) is selected from the group consisting of:

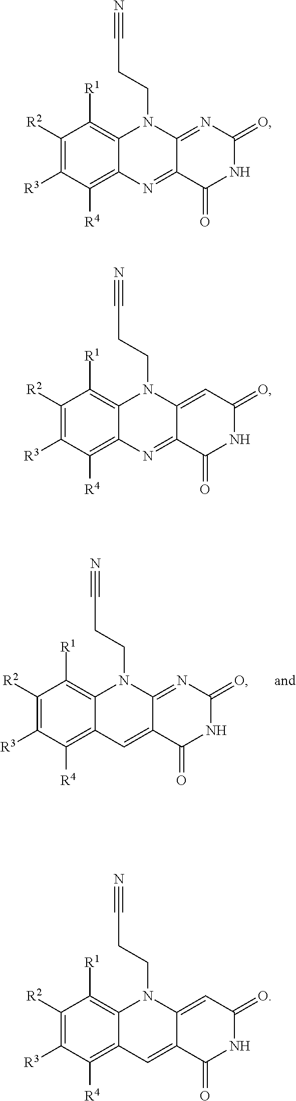

In one aspect, the present disclosure contemplates a compound of Formula (II), or a salt, solvate, tautomer, N-oxide, enantiomer, or diastereoisomer thereof:

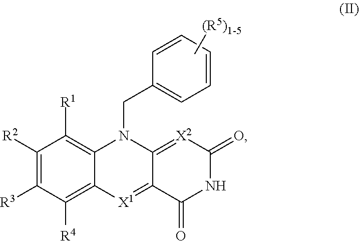

wherein:

X¹ is CH or N;

X² is CH or N;

R¹, R², R³, and R⁴ are independently selected from the group consisting of R, CF₃, OR, OCF₃, C(═O)R, C(═S)R, CN, NO, NO₂, azido, F, Cl, Br, I, N(R)₂, SR, S(═O)R, S(═O)₂R, SO₃R, S(═O)₂N(R)₂, N(R)S(═O)₂R, N(R)S(═O)₂N(R)₂, C(═O)C(═O)R, C(═O)CH₂C(═O)R, C(═O)OR, OC(═O)R, C(═O)N(R)₂, OC(═O)N(R)₂, C(═S)N(R)₂, (CH₂)₀₋₂N(R)C(═O)R, (CH₂)₀₋₂N(R)N(R)₂, N(R)N(R)C(═O)R, N(R)N(R)C(═O)OR, N(R)N(R)C(═O)N(R)₂, N(R)C(═O)R, N(R)C(═O)OR, N(R)C(═S)R, N(R)C(═O)N(R)₂, N(R)C(═S)N(R)₂, N(C(═O)R)—C(═O)R, N(OR)R, C(═NH)N(R)₂, C(═O)N(OR)R, and C(═NOR)R, or two adjacent groups of R¹-R⁴ combine to form methylenedioxy or ethylenedioxy,
or R¹ and R² combine to form an optionally substituted arylene or heteroarylene group (which is fused to the phenyl group to which R¹ and R² are bound) and/or R² and R³ combine to form an optionally substituted arylene or heteroarylene group (which is fused to the phenyl group to which R² and R³ are bound) and/or R³ and R⁴ combine to form an optionally substituted arylene or heteroarylene group (which is fused to the phenyl group to which R³ and R⁴ are bound);

each occurrence of R is independently H or an optionally substituted carbon-based moiety, such as but not limited to optionally substituted alkyl or optionally substituted cycloalkyl,

and

each occurrence of R⁵ is independently optionally substituted C₁-C₆ alkoxy.

In certain embodiments, the compound of formula (II) is selected from the group consisting of:

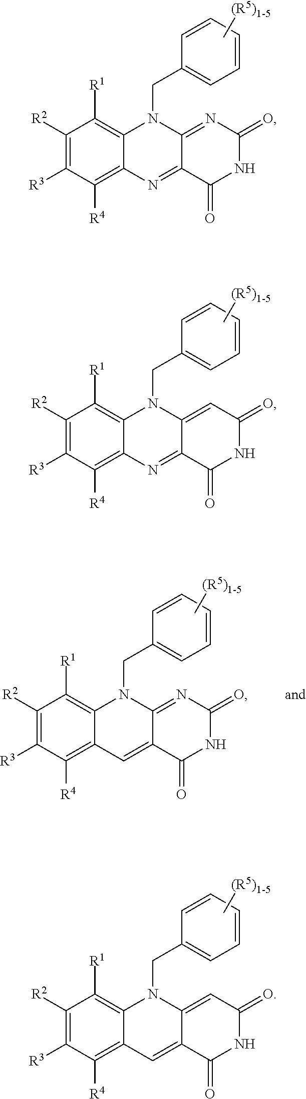

In certain embodiments of (I) and/or (II), R¹ and R² combine to form an optionally substituted arylene or heteroarylene group (which is fused to the phenyl group to which R¹ and R² are bound) and/or R² and R³ combine to form an optionally substituted arylene or heteroarylene group (which is fused to the phenyl group to which R² and R³ are bound) and/or R³ and R⁴ combine to form an optionally substituted arylene or heteroarylene group (which is fused to the phenyl group to which R³ and R⁴ are bound). Thus, ring systems such as substituted naphtho[2,1-g]pteridine-8,10(9H,11H)-dione and/or naphtho[2,3-g]pteridine-2,4(1H,3H)-dione are contemplated within the invention.

Scheme 2 illustrates a non-limiting synthetic scheme contemplated within the disclosure. As demonstrated therein, the derivatization of one of the amino groups of the starting phenylene diamine with the cyanoethyl group (or alkoxy-substituted benzyl group) allows one to achieve specific glycosylation of the resulting alloxazine derivative only on its N3 position. That protecting group can also be easily removed under conditions that do not decompose the molecule. In certain embodiments, deprotection of the cyanoethyl group comprises treatment with base, such as but not limited to alkaline or earth alkaline hydroxide and/or alkaline or earth alkaline methoxide. In certain embodiments, deprotection of the p-methoxy benzyl group comprises treatment with DDQ or hydrogenation using for example Pearlman's catalyst (Pt oxide) and/or Pt on charcoal. The resulting nucleoside/nucleotide can be incorporated in a nucleic acid using standard procedures known in the art.

As illustrated in Scheme 2, the diamine (a) can be reacted with acrylonitrile (vinyl nitrile), in a non-limiting example using a Lewis acid, such as but not limited to aluminum oxide, to form monoderivative (b). Compound (b) can be reacted with alloxan, in a non-limiting example in the presence of boric acid and acetic acid, to form (c). This compound can be reacted with a sugar donor, which in this Scheme is exemplified in a non-limiting manner as (d), such as but not limited to a sugar comprising an anomeric group X selected from halogen, ester (such as but not limited to formyloxy, acetyloxy, benzoyloxy, analogues and derivatives thereof, and the like), amide, sulfide, sulfoxide, sulfone, and the like. This reaction can be performed in the presence of activating agents, such as but not limited to a silylating agent (such as but not limited to BSA) and/or a Lewis acid (such as but not limited to tin chloride), to afford the nucleoside (e). Standard deprotection methods can be used to generate the deprotected compound (f), wherein the sugar is fully or partially deprotected. This compound (f) can then be converted to a 4,4,′-dimethoxytrityl (DMT)-protected diphosphoramidite for incorporation into a nucleic acid using standard nucleic acid coupling conditions.

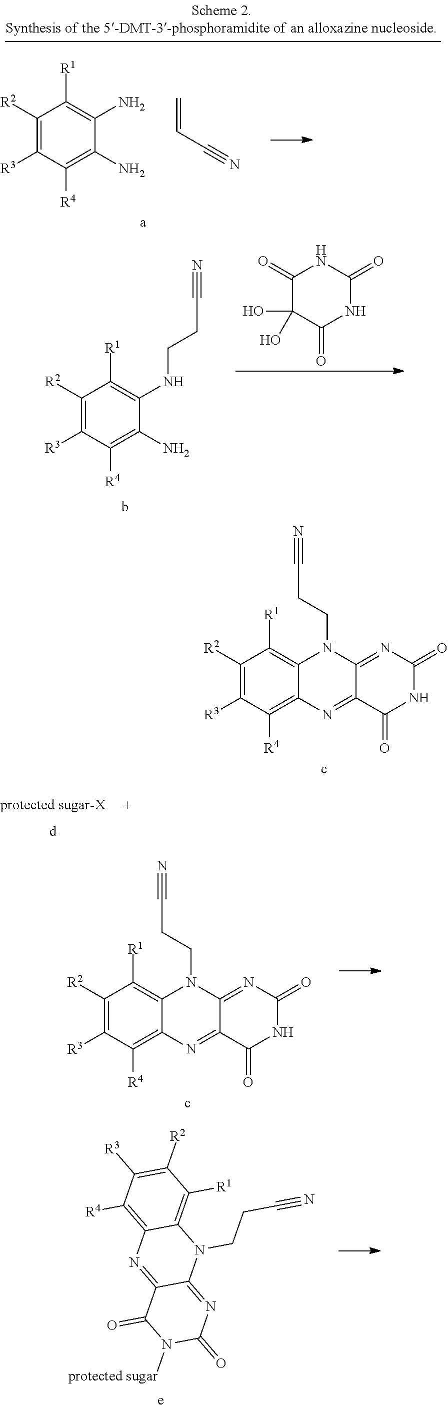

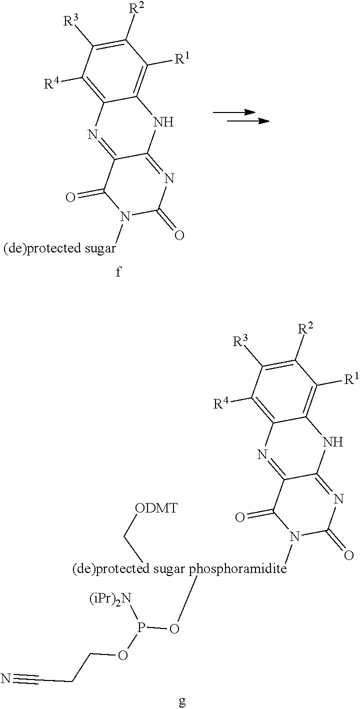

In certain embodiments, the protected activated sugar (d) can be any of the compounds below, wherein:

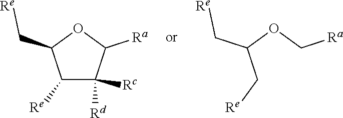

each occurrence of R^{a} is independently a leaving group, such as but not limited to a halogen (such as but not limited to F, Cl, and Br), the ester R^{b}C(═O)O—, the amide R^{b}C(═O)N(H or alkyl)-, mesylate, triflate, an aliphatic or aromatic sulfide, an aliphatic or aromatic sulfoxide, an aliphatic or aromatic sulfone, and the like,
wherein each occurrence of R^{b} is independently optionally substituted alkyl, alkenyl, alkynyl, cycloalkyl, cycloalkenyl, cycloalkynyl, or aryl;

each occurrence of R^{c} is independently F, Cl, optionally substituted C₁-C₁₀ alkyl (such as but not limited to methyl), or optionally substituted C₁-C₁₀ alkoxy (such as but not limited to methoxy),

each occurrence of R^{d} is independently F, Cl, optionally substituted C₁-C₁₀ alkyl (such as but not limited to methyl), or optionally substituted C₁-C₁₀ alkoxy (such as but not limited to methoxy), and

each occurrence of R^{e} is independently a deprotectable group, such as but not limited to R^{f}C(O)O—,
wherein each occurrence of R is independently optionally substituted alkyl, alkenyl, alkynyl, cycloalkyl, cycloalkenyl, cycloalkynyl, or aryl.

In certain embodiments, the DMT can be removed from the nucleoside and/or nucleic acid (such as DNA or RNA) without using acid. In a non-limiting example, the DMT-protected nucleoside and/or nucleic acid (such as DNA or RNA) can be incubated with aqueous dithionite solution (such as for example sodium dithionite, such as for example a 0.2 M sodium dithionite solution, at room temperature for 2 hours), resulting in complete removal of the DMT group. In another non-limiting example, the DMT-protected nucleoside and/or nucleic acid (such as DNA or RNA) can be incubated with aqueous dithionite solution (such as for example sodium dithionite, such as for example a 50-100 mM dithionite solution in aqueous phosphate buffer, pH 5-7, such as but not limited to room temperature, resulting in complete removal of the DMT group.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the disclosure, as well as metabolites and active metabolites of these compounds having the same type of activity. Solvates include water, ether (e.g., tetrahydrofuran, methyl tert-butyl ether) or alcohol (e.g., ethanol), acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds of the disclosure may exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compounds of the disclosure are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, ³³P and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Tomography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

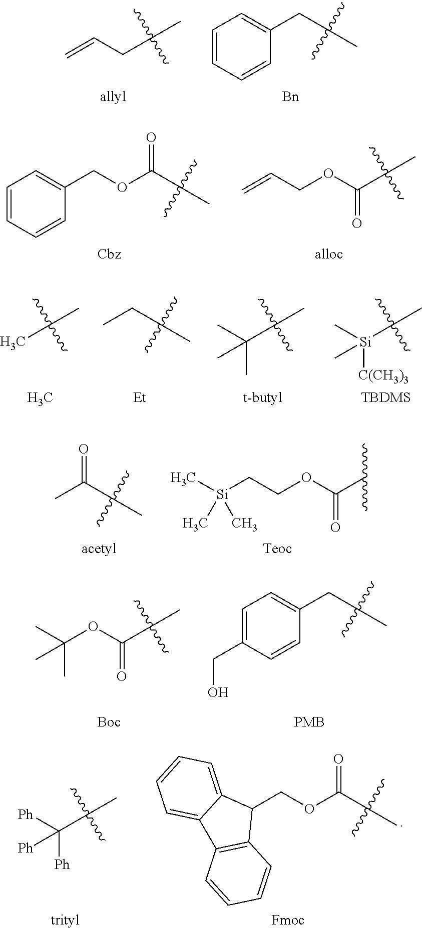

Other protecting groups, plus a detailed description of techniques applicable to the creation of protecting groups and their removal are described in Greene & Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

### Compositions

The disclosure includes a pharmaceutical composition comprising at least one compound of the disclosure and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

### Administration/Dosage/Formulations

The regimen of administration may affect what constitutes an effective amount. The therapeutic formulations may be administered to the subject either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a cancer in the patient. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In certain embodiments, the compositions of the invention are formulated using one or more pharmaceutically acceptable excipients or carriers. In certain embodiments, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the invention are administered to the patient in dosages that range from one to five times per day or more. In other embodiments, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a cancer in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In certain embodiments of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and the progression of a cancer in the patient being treated. The skilled artisan is able to determine appropriate dosages depending on these and other factors.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

### Examples

Various embodiments of the present invention can be better understood by reference to the following Examples which are offered by way of illustration. The present invention is not limited to the Examples given herein.

### Methods and Materials

General. All chemicals were purchased from Sigma-Aldrich unless otherwise noted. Standard reagents used in automated DNA synthesis as well as 5′-dimethoxytrityl-2′-deoxythymidine 3′-[(methyl)-(N,N-diisopropylamino)]-phosphoramidite were purchased from Glen Research. 2′-Deoxynuceosides were purchased from ChemGenes Corporation. Deuterated solvents were purchased from Cambridge Isotopes.

NMR. Experiments were carried out on a Bruker Avance-III 400 (¹H=400.13 MHz). Chemical shifts are given in ppm with positive shifts downfield: all ¹H and ¹³C chemical shifts were referenced relative to the signal from residual protons of a lock solvent. For ¹H 3.31 ppm for MeOD and 5.32 ppm for CD₂Cl₂ and for ¹³C, 49.15 ppm for MeOD and 54.00 ppm for CD₂Cl₂ were used. ³¹P Chemical shifts are referenced to 0.0 ppm in the ¹H NMR spectrum according to the standard IUPAC method.

LC-MS. Analyses were carried out on Waters Synapt G2 HDMS Q-TOF LC/MS spectrometer. A Waters ACQUITY UPLC BEH C18, 1.7 μm, 2.1×100 nm column was used with a gradient of 0-95% of buffer B in 22 minutes with a flow rate of 0.2 mL/min at 40° C. (buffer A was 50 mM triethylammonium acetate buffer pH=7.0 and mobile B was acetonitrile, the following gradient: 0-15 minutes, 0-100% B, 15-18 minutes, 100% B, 18-20 minutes, 100-0% B, 20-22 minutes, 0% B.

HPLC methods. Analytical HPLC injections were carried out on an Agilent Technologies Model HPLC 1100 using an Agilent Hypersil ODS 5 m column, 4.0 mm i.d.×250 mm, eluting at 1.5 mL/min with a gradient of acetonitrile/50 mM triethylammonium bicarbonate buffer, pH 8.0. Preparative HPLC (Agilent Zorbax SB-C18, 5 μm column 9.4 mm i.d.×250 mm) was performed on an Agilent Technologies Model HPLC 1100, eluting at 1.5 mL/min with a gradient of acetonitrile/50 mM triethylammonium bicarbonate buffer. The eluent was monitored for absorption at 254 nm. The same mobile phases and flow rate were applied on both systems; the following gradient: 0-30 minutes, 0-50% B, 30-50 minutes, 50-100% B, 50-60 minutes, 100-0% B, 60-70 minutes, 0% B.

Thermal Melting Experiments. Duplex melting experiments were performed on a Cary 100 Bio UV-visible spectrophotometer (Varian, Santa Clara, Calif.) equipped with a thermoelectrically controlled multicell holder. Samples contained 1 μM alloxazine-DNA and complementary DNA or RNA in solutions of 1.0 M NaCl/0.01 M Na₂HPO₄, 0.10 M NaCl/0.01 M Na₂HPO₄, or 0.01M NaCl/0.01 M Na₂HPO₄ at pH 7.3. The samples were heat denatured at 90° C. for 10 min, cooled to 25° C. at a rate of 1° C./min, and maintained at this temperature for 10 min. Duplex melting was performed by heating the duplexes from 25 to 90 at 1° C./min with the absorbance (260 nm) being recorded at one-minute intervals. Melting temperatures were determined at the maximum of first derivative plots. In order to confirm the stability of duplexes and the effect of the heating/cooling cycles, the melting temperatures were measured four times for each sample (twice during the heating cycle and twice during the cooling cycle).

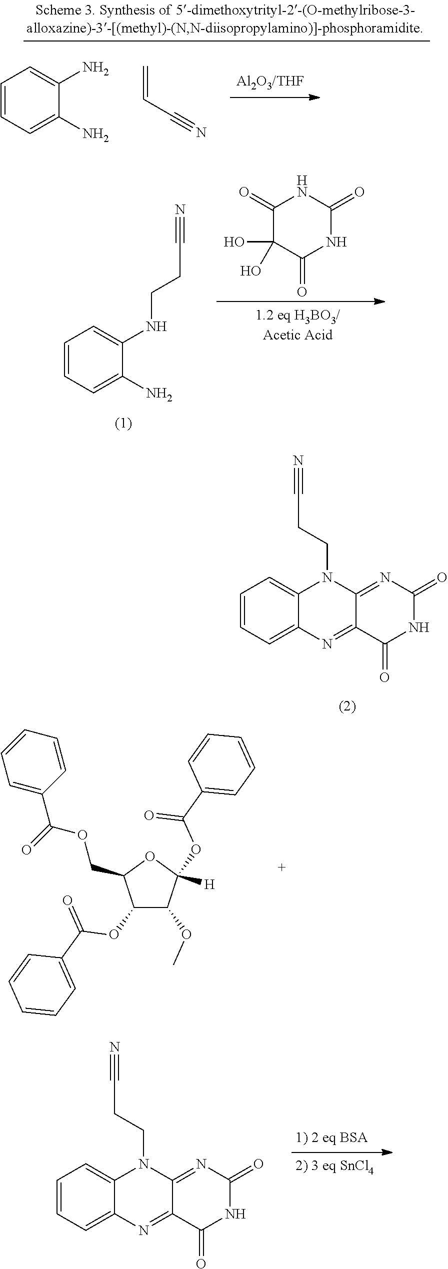

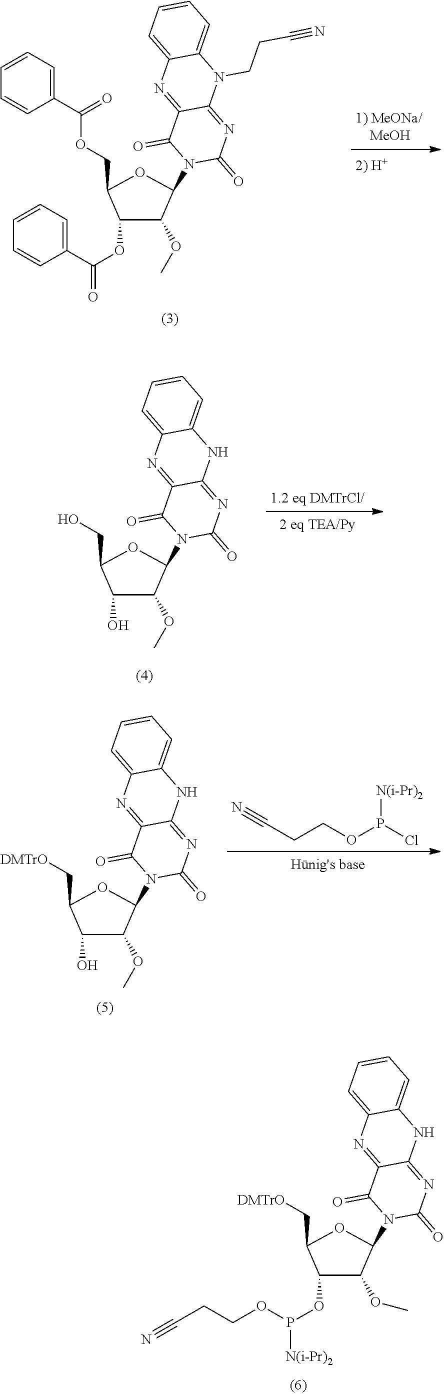

### Example 1: Synthesis (Scheme 3)

### Synthesis of 3-(2-Aminophenylamino)propanenitrile (1)

A mixture of o-phenylenediamine (1 mmol), acrylonitrile (1.5 mmol), and Al₂O₃ (1 g) was dissolved in THE and refluxed under argon for 24 hours. The reaction mixture was cooled to room temperature and filtered, the solid mixture was washed with ethyl acetate (10 mL), and the crude product in the filtrate was obtained after removing off the ethyl acetate/THF from washing solution. The crude mixture was purified on silica column using (ethylacetate/hexane 1:1) as eluent. Yield 62%.

### Synthesis of N-10-(4-propanylnitrile) alloxazine (2)

In a 50 mL round bottom flask, 3-(2-Aminophenylamino)propanenitrile (1) (3.0 mmol), alloxane monohydrate (0.5 g, 3.0 mmol), boric acid (0.3 g, 3.0 mmol), and 20 mL acetic acid were added. The mixture was stirred under argon at room temperature for 24 hours. During this time, solid precipitated out in the solution. The solid was collected by filtration then washed with 10 mL of acetic acid and 30 mL of diethyl ether. Product was dried further using a high vacuum pump. Yield 86%.

### Synthesis of 1-(N-3-(N-10-(4-propanylnitrile)alloxazinyl)-3,5-di-O-benzoyl-2-O-methyl-α-D-Ribose (3)

A solution of N-10-(4-propanylnitrile) alloxazine (2) (1.5 g, 5.5 mmol) in dry acetonitrile (25 mL) under argon was cooled to 0° C., and (2.28 g, 11 mmol) of N,O-bis(trimethylsilyl)acetamide (BSA) was added dropwise over 10 minutes. The reaction mixture was stirred for 2 hours and 1,3,5-Tri-O-benzoyl-2-O-methyl-α-D-ribose (2.4 g, 5 mmol) was added and stirred for additional 15 minutes. Tin tetrachloride (4.0 g, 15 mmol) was added and stirred overnight. Triethylamine (25 mL) was added, followed by 200 mL of water, and the reaction mixture was stirred for 15 minutes until all the tin precipitate disappeared. Then the system poured into saturated sodium bicarbonate solution (20 mL) and extracted with ethyl acetate (3×150 mL). The combined organic extracts were dried over magnesium sulfate, filtered, and evaporated. Purification by flash chromatography on silica, eluting with ethylacetate, gave 3 (1.4 g, 45% yield) as a yellow solid.

### Synthesis of 1-(N-3-alloxazinyl)-2-O-methyl-α-D-Ribose (4)

To a stirred solution of (3) (2.8 g, 4.5 mmol) in anhydrous methanol (40 mL) under argon, 4 mL of 25% methanolic sodium methoxide was added and stirred for 4 hours, then the system was neutralized with Dowex 50 (WX8, H+ form) ion exchange resin. The resin was removed by filtration and washed with methanol. The filtrate was evaporated under reduced pressure to give 4 (1.45 g, 90% yield) as a yellow solid.

### Synthesis of 1-(N-3-alloxazinyl)-2-O-methyl-5-O-(4,4′-dimethoxytrityl)-α-D-Ribose (5)

1.8 g (5 mmol) of (4) was suspended in 50 mL of dry pyridine under argon. 2 g (6 mmol) of 4,4′-dimethoxytritylchloride and 1.4 mL (10 mmol) of triethylamine were added, and the reaction mixture was stirred overnight. The mixture was then poured into 250 mL of cold, saturated NaHCO, and the solution was extracted three times with 150-mL portions of ethylacetate. The combined organic layers were evaporated to dryness and purified by flash chromatography on a silica column. The desired product was eluted using ethylacetate and isolated as a yellow foam. (3.0 g-90% yield).

### Synthesis of 1-(N-3-alloxazinyl)-3-O-(cyanoethyl-N,Ndiisopropylphosphoramidite)-2-O-methyl-5-O-(4,4′-dimethoxytrityl)-α-D-Ribose (6)

Compound (5) (3 g, 4.5 mmol) was dried overnight under vacuum in a round bottomed flask. The flask was then flushed repeatedly with argon. Anhydrous dichloromethane (50 mL), N,N-diisopropylethylamine (1.2 mL, 6.8 mmol), and N,Ndiisopropylamino-cyanoethylphosphoamidic chloride (1.3 g, 5.5 mmol) were added to the flask. The reaction mixture was stirred at room temperature for one hour under argon. At this time TLC (7:3; ethylacetate:hexanes) showed complete disappearance of starting material. The organic solvent was evaporated to dryness. The crude product was dissolved in a mixture of ethylacetate:hexane (1:1) and purified by flash chromatography on a silica column. The silica gel slurry was prepared with the starting eluant mixture containing an additional 5% triethylamine. After pouring the silica gel slurry, the column was washed with two column volumes of the starting solvent mixture containing no triethylamine. Compound (6) was purified using a gradient of ethylacetate:hexanes (1:1) to ethylacetate:hexanes (7:3) and isolated as a yellow foam (2.3 g, 61%).

### Example 2: Solid-Phase Synthesis

Low Volume controlled pore glass (CPG) columns (1 μmol synthesis scale) were purchased from Glen Research. DNA synthesis was carried out on an Applied Biosystems Model 394 automated DNA synthesizer (Applied Biosystems) using the standard DNA synthesis cycle when regular phosphate linkage with synthesized. Other reagents were: Activator (0.25 M ETT), Cap A (THF/Pyridine/Ac₂O), Cap B (16% 1-methylimidazole in THF) were purchased from Glen Research.

### Solid-Phase Synthesis for Phosphoramidate

Monomer amidites (such as 6) was dissolved in anhydrous acetonitrile at 0.15M concentration. The solution was delivered to the column using ports 5-7 with 0.25M 5-(Ethylthio)-1H-tetrazole (ETT) in acetonitrile. Coupling time was 15 minutes. Table 1 summarizes the solid phase synthesis conditions.

TABLE 1
Solid phase synthesis cycle for phosphoramidate.

| Step | Reaction Step | Reagent/Conditions | Time |
|---|---|---|---|
| 1 | Detritylation | 3% Trichloroacetic acid in DCM | Flow 95 s |
| 2 | Wash | Acetonitrile | Flow 30 s |
| 3 | Condensation | 0.15M phosphoramidte in | 900 s wait |
| | | acetonitrile with | |
| | | 0.25 ETT in acetonitrile | |
| 4 | Capping | Cap A (THF/Pyridine/Ac₂O), | Flow 10 s, |
| | | and Cap B (16% 1-methylimidazole | wait 5 s |
| | | in THF) | |
| 5 | Oxidatiation | 0.02M Iodine in THF/Water/Pyridine | 20 s |
| 6 | Wash | Anhydrous acetonitrile | Flow 60 s |
| 7 | Wash | DCM | Flow 35 s |

After completion of solid-phase synthesis, oligonucleotides were cleaved from the CPG solid support and the heterocyclic bases deprotected by incubated in 0.05M potassium carbonate in methanol for 12 hours at RT. The solution was neutralized using 7 uL glacial acetic acid and evaporated using speed vacuum. Reconstituted in water at pH 8 and filtered from the solid support. The crude oligonucleotide applied to analytical HPLC to check purity or semi-preparative RP-HPLC column for purification.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A compound of formula (III), or a salt, solvate, tautomer, N-oxide, enantiomer, or diastereoisomer thereof: 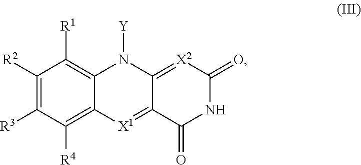
wherein:
Y is —CH₂CH₂CN or 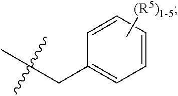
X¹ is CH or N;
X² is CH or N;
R¹, R², R³, and R⁴ are independently selected from the group consisting of R, CF₃, OR, OCF₃, C(═O)R, C(═S)R, CN, NO, NO₂, azido, F, Cl, Br, I, N(R)₂, SR, S(═O)R, S(═O)₂R, SO₃R, S(═O)₂N(R)₂, N(R)S(═O)₂R, N(R)S(═O)₂N(R)₂, C(═O)C(═O)R, C(═O)CH₂C(═O)R, C(═O)OR, OC(═O)R, C(═O)N(R)₂, OC(═O)N(R)₂, C(═S)N(R)₂, (CH₂)₀₋₂N(R)C(═O)R, (CH₂)₀₋₂N(R)N(R)₂, N(R)N(R)C(═O)R, N(R)N(R)C(═O)OR, N(R)N(R)C(═O)N(R)₂, N(R)C(═O)R, N(R)C(═O)OR, N(R)C(═S)R, N(R)C(═O)N(R)₂, N(R)C(═S)N(R)₂, N(C(═O)R)—C(═O)R, N(OR)R, C(═NH)N(R)₂, C(═O)N(OR)R, and C(═NOR)R, or two adjacent groups of R¹-R⁴ combine to form methylenedioxy or ethylenedioxy;
or R¹ and R² combine to form an optionally substituted arylene or heteroarylene group (which is fused to the phenyl group to which R¹ and R² are bound) and/or R² and R³ combine to form an optionally substituted arylene or heteroarylene group (which is fused to the phenyl group to which R² and R³ are bound) and/or R³ and R⁴ combine to form an optionally substituted arylene or heteroarylene group (which is fused to the phenyl group to which R³ and R⁴ are bound);
each occurrence of R is independently H, optionally substituted alkyl, or optionally substituted cycloalkyl; and
each occurrence of R⁵ is independently optionally substituted C₁-C₆ alkoxy.

2. The compound of claim 1, wherein at least one R⁵ is methoxy.

3. The compound of claim 1, which is selected from the group consisting of:

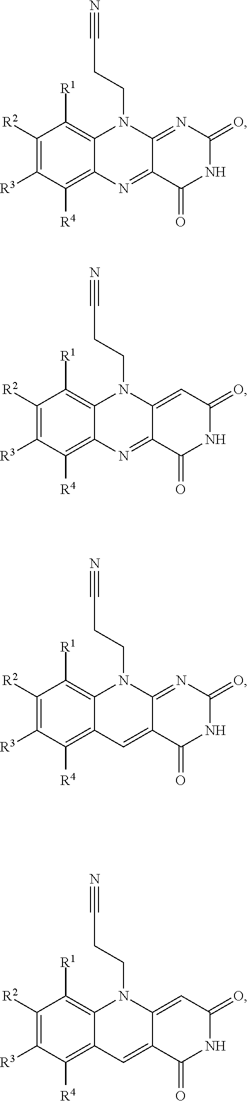

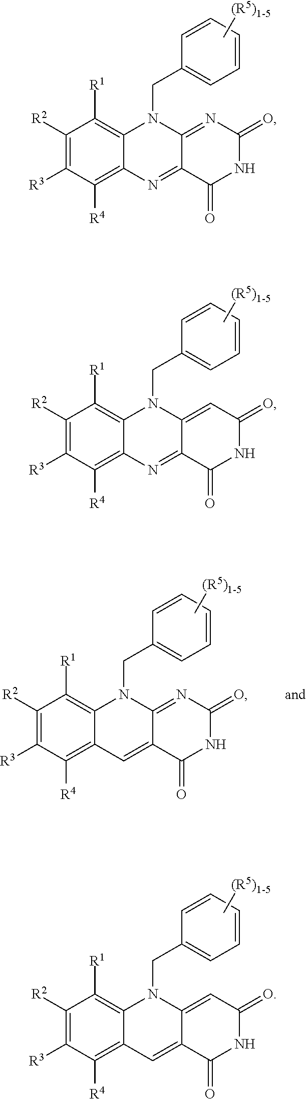

4. The compound of claim 1, wherein R¹, R², R³, and R⁴ are H.

5. A method of preparing a derivatized nucleoside, the method comprising reacting the compound of claim 1 with a sugar donor comprising an anomeric leaving group.

6. The method of claim 5, wherein the anomeric leaving group is a halogen, ester, amide, mesylate, triflate, sulfide, sulfoxide, or sulfone.

7. The method of claim 5, wherein the anomeric leaving group is an ester and the reaction is performed in the presence of a silylating agent and a Lewis acid.

8. The method of claim 5, wherein the sugar is a ribose or ribose derivative.

9. The method of claim 5, wherein the sugar is a deoxyribose or deoxyribose derivative.

10. The method of claim 5, wherein the sugar donor is one of the following: 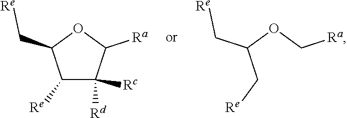
wherein:
each occurrence of R^{a} is independently a leaving group selected from a halogen, the ester R^{b}C(O)O—, the amide R^{b}C(O)N(H or alkyl)-, mesylate, triflate, an aliphatic or aromatic sulfide, an aliphatic or aromatic sulfoxide, and an aliphatic or aromatic sulfone,
wherein each occurrence of R^{b} is independently optionally substituted alkyl, alkenyl, alkynyl, cycloalkyl, cycloalkenyl, cycloalkynyl, or aryl;
each occurrence of R^{c} is independently F, Cl, optionally substituted C₁-C₁₀ alkyl, or optionally substituted C₁-C₁₀ alkoxy,
each occurrence of R^{d} is independently F, Cl, optionally substituted C₁-C₁₀ alkyl, or optionally substituted C₁-C₁₀ alkoxy, and
each occurrence of R^{e} is independently a deprotectable group.

11. The method of claim 5, wherein the resulting derivatized nucleoside is subjected to deprotecting conditions that allow for replacement of Y with a hydrogen.

12. The method of claim 11, wherein Y is —CH₂CH₂CN and the deprotecting conditions comprise treatment with a base.

13. The method of claim 11, wherein Y is 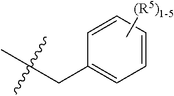
and the deprotecting conditions comprise treatment with an oxidizing agent.

14. The method of claim 5, wherein the derivatized nucleoside is further converted to a phosphoramidite derivative.

15. A nucleic acid wherein at least one base is: 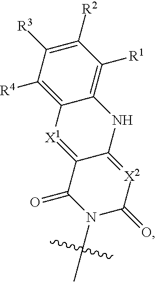
wherein:
X¹ is CH or N;
X² is CH or N;
R¹, R², R³, and R⁴ are independently selected from the group consisting of R, CF₃, OR, OCF₃, C(═O)R, C(═S)R, CN, NO, NO₂, azido, F, Cl, Br, I, N(R)₂, SR, S(═O)R, S(═O)₂R, SO₃R, S(═O)₂N(R)₂, N(R)S(═O)₂R, N(R)S(═O)₂N(R)₂, C(═O)C(═O)R, C(═O)CH₂C(═O)R, C(═O)OR, OC(═O)R, C(═O)N(R)₂, OC(═O)N(R)₂, C(═S)N(R)₂, (CH₂)₀₋₂N(R)C(═O)R, (CH₂)₀₋₂N(R)N(R)₂, N(R)N(R)C(═O)R, N(R)N(R)C(═O)OR, N(R)N(R)C(═O)N(R)₂, N(R)C(═O)R, N(R)C(═O)OR, N(R)C(═S)R, N(R)C(═O)N(R)₂, N(R)C(═S)N(R)₂, N(C(═O)R)—C(═O)R, N(OR)R, C(═NH)N(R)₂, C(═O)N(OR)R, and C(═NOR)R, or two adjacent groups of R¹-R⁴ combine to form methylenedioxy or ethylenedioxy;
or R¹ and R² combine to form an optionally substituted arylene or heteroarylene group (which is fused to the phenyl group to which R¹ and R² are bound) and/or R² and R³ combine to form an optionally substituted arylene or heteroarylene group (which is fused to the phenyl group to which R² and R³ are bound) and/or R³ and R⁴ combine to form an optionally substituted arylene or heteroarylene group (which is fused to the phenyl group to which R³ and R⁴ are bound);
each occurrence of R is independently H, optionally substituted alkyl, or optionally substituted cycloalkyl.

16. The nucleic acid of claim 15, wherein the nucleic acid is a ribonucleic acid.

17. The nucleic acid of claim 15, wherein the nucleic acid is a deoxyribonucleic acid.
